(19) United States
(12) Patent Application Publication
(10) Pub. No.: US 2018/0318384 A1
(43) Pub. Date: Nov. 8, 2018

(54) Strengthened t-cell modulator that can modulate the immune response, specifically designed for therapeutic use for the treatment of the disease known as vitiligo

(71) Applicant: HECTOR MANUEL ZEPEDA LOPEZ, Tlalnepantla (MX)

(72) Inventor: HECTOR MANUEL ZEPEDA LOPEZ, Tlalnepantla (MX)

(21) Appl. No.: 15/579,534

(22) PCT Filed: Jun. 4, 2015
(86) PCT No.: PCT/MX2015/000086

§ 371 Date: Dec. 4, 2017

(51) Int. Cl. A61K 38/17; A61K 35/60; A61K 35/15; A61K 9/14; A61P 37/02

(52) U.S. Cl. CPC A61K 38/1706; A61K 35/60; A61K 38/02; A61K 9/14; A61P 37/02; A61K 35/15

(57) ABSTRACT

The invention relates to a strengthened T-cell modulator with a strength of 10¹² leukocytes/mm³, produced from a dialysed extract of leukocytes from the spleen of selachimorpha or sharks, which contains a maximum of 10,000 Da., in the form of a powder. The invention also relates to the use thereof for producing a medicament for treating the disease known as vitiligo.

The present invention refers to a leukocyte extract containing polypeptides equal to or less than 10,000 daltons from shark spleen, to obtain a pontentited T-cell modulator (TCM for its acronym in English) capable of modulating the immune response through the activation of specific molecules involved in the control of innate immunity called “Toll-like Receptors” and its use as a pharmaceutically acceptable composition for the treatment of the disease known as vitiligo.

### BACKGROUND

Vitiligo is a degenerative disease of the skin in which melanocytes (the cells responsible for pigmentation of the skin) die, with which the production of melanin (the substance producing the skin pigmentation) is stopped in the area where the cell death has occurred.

In most cases it begins between the 10 and 30 years and is manifested by the appearance of white spots which result from the absence of pigment in the skin. In principle they are usually circular areas with defined edges and with a variable extension that is usually observed more frequently in the extremities (hands and feet), areas of extension and flexion (knees and elbows), the face or the genitals. The depigmented areas with time can grow and spread to any other part of the body. Vitiligo is not contagious, neither by touch nor by any other type of contact, since the processes that trigger it are inherent in the person. Its consequences are slight: it increases the susceptibility to sunburn in areas without pigmentation and mainly causes aesthetic problems.

The prevalence of this condition is between 0.5 and 3% of the population. There are no differences by sex or race.

The causes of the onset of this disease have not yet been fully elucidated and the mechanisms by which this alteration is unleashed are still under study, although it seems that in 30-40% of cases there is a family history of this pathology that would be inherited through a multifactorial polygenic model with variable expression.

There are three theories that try to explain the etiopathogenic mechanism:

The autoimmune theory: Melanocytes are destroyed by certain activated lymphocytes. It would be similar to what happens in other better known autoimmune processes (such as some thyroiditis), and is confirmed by the response of some cases to treatments with immunosuppressive drugs.

Neurogenic theory: A possible interaction between melanocytes and nerve cells is postulated which would release a toxic neurochemical mediator. This would be the cause of the destruction of melanocytes.

The theory of self-destruction: According to this proposal melanocytes would be destroyed by toxic substances formed in the metabolic processes of biosynthesis of the melanin (through certain metabolic pathways active only in some subjects).

However, recently there have been studies that have shown that vitiligo is caused by the increase of the amount of adrenaline in the bloodstream that produces an overload in the functioning of the spleen, liver, kidney and pancreas, by the excess of toxins (free radicals) that they are not able to eliminate. These toxins are accumulated in the liver and in all those organs responsible for their elimination, and this leads to the melanocytes remaining undifferentiated in the basal layer as a consequence of the lack of blood flow, leading the melanocytes to lose their functions and remain intact in the basal zone as undifferentiated cells (that is, without the function of creating melanin, which is the substance that gives color to the skin) and, added to this, the inability of keratinocytes to retain the little melanin produced by the melanocytes. As part of the accumulation of these residues in the liver, the symptoms that are usually present in these patients, such as headaches, abdominal pain, reluctance, depression, and itching in areas of the lesions, hearing loss (all of them can be present as just some of them).

One of the new proposals for the management of vitiligo and other diseases of autoimmune origin is the dialysed leukocyte extract (DLE), which was described in 1955 by S. Lawrence when he demonstrated that leukocyte dialysate from a healthy donor reactive to tuberculin could transfer, to another healthy donor, the ability to respond to said test. The molecular description of the dialysed leukocyte extract was made up to 1992 by Kirkpatrick.

It is now known that DLE is a set of low molecular weight peptides less than 10 kDa. The first animal models were made in mice and cows which were immunized with different antigens, including glycoprotein D of the simplex herpes virus, and the antigen-specific DLE used to transfer immunity to other animals was purified from their blood.

These effects are due to the ability to transfer a specific antigen immunocellular response to non-immune receptors, as well as to increase the number of immunocompetent cells and to stimulate the phagocytosis and hematopoiesis through the induction of the cytokine synthesis such as interleukin 2 (IL-2), interferon gamma (INF-g) and tumor necrosis factor alpha (TNF a).

In other investigations it was shown that the production of osteopontin, cytokine that exerts antagonistic effects on the immune response depending on its concentration (at low concentrations stimulates the immune response and vice versa), decreased.

In summary, DLE has direct effects on cell activation, signaling, activation of transcription factors, genes and cytokines. Its properties go in two directions: as inducers of the immune response (not antigen-specific and specific antigen) and as suppressors (avoiding the over-response or autoimmunity) probably increasing the action of regulatory T cells and decreasing the effectors.

The DLE can be obtained from the blood or lymphoid tissue of different animal species, given that since 1975 it was demonstrated that its effect is not species-specific. For its use in humans we find DLE obtained from blood of healthy donors, bovine colostrum, and egg yolk.

In the year of 1955, Lawrence, interested in specific active immune memory, found that this could be transferred from one person to another by means of a leukocyte extract injection to which he called ‘Transfer Factor’.

Transfer Factor: leukocyte dialyzed extract constituted by a group of molecules of low molecular weight between 1.0 and 6.0 KDa; said molecules store the exclusive immune experience of the animal which, in turn, can be transferred to the human.

However, in the prior art there is no means for obtaining a dialyzable extract of leukocytes containing polypeptides less than or equal to 10,000 Daltons whose source or origin is from cells, tissues or organs of sharks, more specifically shark spleen, like that of the present invention, which is considered novel, since when analyzing the current prior art, methods were found for said extraction of leukocytes, white cells or T cell modulator, by means of leukocytic packages of healthy donors (human); eggs of reptiles, amphibians, fish and birds; besides colostrum (milk produced by mammal) and crocodile spleen. But no document was found mentioning the possibility of extracting leukocyte cells from selacimorphs for the extraction of TCM, which are a superorder of chondrichthyes (cartilaginous fish) commonly known as sharks, wherein it has been demonstrated in in vitro tests to be eight times more potent than the peripheral blood derivative of healthy donors in the induction of cytokines, specifically IL-6, IFN-y TNF-a.

In this sense, the concept about the immunological capacity in ancestral animals is: The longer age the immune system is better, which allows to survive so many years, so when giving us the task of obtaining spleen cells from an ancestral animal it was found that the Shark is an excellent candidate to obtain spleen cells that can be used to obtain a higher potency T-cell modulator.

Therefore, numerous clinical trials have been carried out in the last 3 decades using extracts of white blood cells known as “transfer factor”. Unfortunately, the different scientific groups working in this field were not able to consistently generate purified and reproducible preparations of the transfer factors. Despite this, the efficacy of the transfer factors have been published in revised publications for the treatment of conditions ranging from multiple sclerosis, cancer, and herpes infections but none for the treatment of vitiligo.

In this sense, the inventors of the present invention were able to develop an economic and scalable protocol for the generation of an optimized TCM from shark leukocytes. Unlike previous versions of transfer factors, TCM is characterized at molecular level and its main means of inducing therapeutic effects for the treatment of vitiligo have been elucidated.

According to the previous, it is evident that at present there is no an effective treatment available against the destruction of the melanocytes that occurs in vitiligo. Repigmentation has been attempted through the use of steroids or immunomodulators (topical and systemic) with poor results, the psoralens in combination with ultraviolet light sessions are also used with limited efficacy, all of these without achieve fully satisfactory results.

Therefore, in the prior art there is no source that provides a dialyzable extract of leukocytes containing polypeptides less than or equal to 10,000 Daltons whose source or origin is from cells, tissues or organs of sharks with a concentration of T cells modulator of around 10¹² leukocytes×mm³ for use in the treatment of vitiligo. Since, in said prior art, the T cell modulator concentrations of the known extraction media, ranges from 10⁴ to 10⁸ leukocytes×mm³.

### OBJECTS OF THE INVENTION

Therefore, it is an object of the present invention to provide an effective medical treatment which activates the production of melanocytes to normalize melanin levels in the body for the treatment of vitiligo.

It is also an object of the present invention to provide a potentiated T cell modulator with a concentration of approximately 10¹² leukocytes×mm³, and its pharmaceutical use specifically designed for the treatment of vitiligo.

Still another additional object of the present invention is to provide a potentiated T cell modulator with a concentration of approximately 10¹² leukocytes×mm³ which is capable of providing effective immune regulation in the treatment of vitiligo.

A further object of the present invention is to provide a potentiated T cell modulator with a concentration of approximately 10¹² leukocytes×mm³ that is capable of carrying out a retrogression of the disease known as vitiligo.

### BRIEF DESCRIPTION OF THE FIGURES

FIG. 1 shows a graph illustrating the stage in which the test subjects were in the initial state.

FIG. 2 shows a graph that illustrates the stage in which the test subjects were in the last year.

FIG. 3 shows a graph that illustrates which test subjects presented or not dehiscence.

FIG. 4 shows a graph that illustrates which test subjects presented or not erythema.

FIG. 5 shows a graph illustrating test subjects who were withdrawn from the study because of Hypertransaminasemia.

FIG. 6 shows a graph that illustrates the test subjects who left the study by their own.

FIG. 7 shows a graph that illustrates the final result of the test subjects who finished the study.

FIG. 8 shows the three-dimensional structure of conserved sequence.

### DETAILED DESCRIPTION OF THE INVENTION

The present invention relates to a potentiated T-cell modulator (TCM) which is specifically obtained for the treatment of the symptomatology of the disease known as vitiligo, said TCM has the ability to reactivate the cells, specifically the system immunological of the individual, which provides the ability of the body to reactivate the cells responsible for pigmentation of the skin, ie the melanocytes, and thus balancing the production of melanin in the skin by said cellular activation.

In studies performed on patients with vitiligo disease, it was found that by consuming the potentiated T-cell modulator, around 90% resulted in significant improvements, stopping the advancement of skin depigmentation in the first months, to subsequently star to produce islands of pigment in the acromic spots, which results in a regeneration of the depigmented parts around month 6.

The Potentialized T-cell Modulator specifically designed for pharmaceutical use in the medical treatment of the vitiligo of the present invention, helps neurotransmitters to re-establish chemical signaling that travels from the brain to the bone marrow by coupling to the chemical signals of the neurotransmitters for whereby potentiate the signal, which manages to establish the correct chemical signaling to the melanocytes and whereby “order” them to start producing melanin. In this way, the melanocytes are forced to restore the correct levels of melanin production to achieve pigmentation of the depigmented areas due to the low amount of melanin and therefore to cause the regression of vitiligo disease.

This means that with the continuous administration of the T-cell modulator of the present invention, the typical symptomatology of the disease known as vitiligo can be eliminated, since by assisting the trip of chemical signals from the brain to the bone marrow, it is ordered the production of white blood cells, which are the raw material of the production of melanocytes and these in turn are responsible for producing melanin, which is the latter the deficiency found in the skin of patients with the disease known as vitiligo.

Likewise, as it is well known for a person skilled in the art, that the T-cell modulator has as its main function to aid in the production and correct restoration of white cells and the excitation thereof, in order to achieve the optimal functioning of the immune system. Likewise, it is therefore understandable that the potentiated T-cell modulator with a concentration of approximately 10¹² leukocytes×mm³of the present invention, effectively helps the treatment of the disease known as vitiligo, since it is provided the possibility of alleviate the symptomatology characteristic of this, by cellular excitement of the immune system of the individual and proper functioning of the neurotransmitters, which are the chemical signals responsible for various functions of the body, including the production of white blood cells or leukocytes which in turn translate among other things in melanocytes and in turn melanin.

As mentioned above, the potentiated T-cell Modulator for the treatment of the disease known as vitiligo of the present invention, consists of a potency of 10¹² leukocytes×mm³ (meaning by potency the amount of leukocytes and quality of the smooth, round and innocuous cells), amount necessary for the excitation of the leukocytes and optimization of the chemical signals for the production of white blood cells, which in turn produce melanocytes and the latter melanin. It should be noted that with a T-cell modulator concentration lower than that established in the present invention, it would not be possible to treat the symptomatology of the disease known as Vitiligo, since it would not have sufficient power to achieve the production of chemical signals that act on leukocytes which in turn induce the production of melanocytes and the latter melanin.

Therefore the potentiated TCM of the present invention is specifically designed for the treatment of the disease known as vitiligo by administering the cytosine(s) suitable for the activation of melanocytes that will produce melanin and thus repigment the depigmented zones by the lack of melanin. The TCM of the present invention is in powder form, which provides the virtue of being easily transported and stored, does not require refrigeration and has a power of 10¹² leukocytes×mm³, which is highly superior to any known T cell modulator, being understood by power to the concentration of leukocytes per mm³ and the quality of the cells (smooth, round and innocuous).

Biotechnological products containing said powder of TCM represent a very important therapeutic advance, due to their immunomodulatory characteristics, not interfering with traditional treatments for certain diseases.

In the case of powder of TCM, it has the T-cell modulator molecules necessary to regulate the immune response that develops in vitiligo, which is the action of cytotoxic T-lymphocytes against melanocytes.

The regulatory action of the cytotoxic T-lymphocytes that act against the melanocytes, causes the reactivation of the melanocytes in such a way that the patient initiates the pigmentation of the area that was depigmented by the vitiligo.

The immediate appearance of erythema in the depigmented area indicates the action of proinflammatory cytokines (IL-2, IFNγ and TNFα), initiating a chain reaction of chemical signals which lead to the regulation of the immune system, especially cytotoxic T-Iymphocytes which act on melanocytes.

For a better understanding of the invention, illustrative examples of the use and application thereof are shown below.

### Evidence 1

Protocol “Study of Phase II, Open, to Evaluate the Safety and Response of the T Cell Modulator (Ch11001hz) in Patients with Vitiligo”.

23 subjects were evaluated for admission to the protocol entitled “Study of Phase Open, to Evaluate the Safety and Response of the T Cell Modulator (Ch11001hz) in Patients with Vitiligo”, from which 3 subjects were selection failure (screening failure) due to the following causes: transaminase elevation 3 times higher than the normal upper limit, due to chronic decompensated disease (Diabetes Mellitus 2) with acute and chronic complications and a history of uncontrolled psychiatric illness.

We enrolled (randomized) 20 subjects of which 13 (65%) were women and 7 (35%) were men. From the 20 subjects, 8 (40%) had a family history of vitiligo; this finding is different from what is usually reported in the literature where a family relationship less than 20% is mentioned.

History of smoking in 7 subjects (35%) and 5 subjects (30%) had a history of alcoholism shown in the following Table.

TABLE 1

| | n | % |
|---|---|---|
| Patients | 23 | 115 |
| Screen Failure | 3 | 15 |
| Patients of the study | 20 | 100 |
| Female | 13 | 65 |
| Male | 7 | 35 |
| History of vitiligo | 8 | 40 |
| Smoking | 7 | 35 |
| Alcoholism | 5 | 30 |

The age range of the subjects was 18-65 years, with a median of 38.5 years; the evolution time of vitiligo in a range of 2-42 years with a median of 13 years; the weight in a range of 45.5-100.7 kg, with a median of 66.2 kg; the initial vitiligo body surface with a range of 8-59.5% and a median of 31.5%, which is shown in the following Table.

TABLE 2

| | |
|---|---|
| Median | Rank |

| | | | |
|---|---|---|---|
| Age | 38.5 | años | 18-65 |
| Vitiligo Evolution time | 13 | años | 2-42 |
| Weight | 66.2 | kg | 45.6-100.7 |

| | | |
|---|---|---|
| Initial Vitiligo Body Surface | 31.5% | 8-59.5 |

The marital status of the subjects was: single 9 subjects (45%), married 8 subjects (40%), divorced 2 subjects (10%) and widower 1 subject (5%). (See Table 3)

TABLE 3

| Marital Status | n | % |
|---|---|---|
| Single | 9 | 45 |
| Married | 8 | 40 |
| Divorced | 2 | 10 |
| Widower | 1 | 5 |

In relation to chronic degenerative diseases, 3 subjects (15%) presented arterial hypertension, another 1 subject (5%) with Diabetes Mellitus type 2, 2 subjects with other pathologies and 14 subjects (70%) did not present any chronic degenerative disease (See Table 4).

TABLE 4

| Chronic Degenerative Diseases | n | % |
|---|---|---|
| Arterial Hypertension | 3 | 15 |
| Other | 2 | 10 |
| Diabetes Mellitus Type 2 | 1 | 5 |
| None | 14 | 70 |

Occupation: Employee 7 subjects (35%), Student 5 subjects (25%), worker of medical area 3 subjects (15%), housewife 2 subjects (10%), other 2 subjects (10%), merchant 1 subject (5%). (See Table 5).

TABLE 5

| Occupation | n | % |
|---|---|---|
| Employee | 7 | 35 |
| Student | 5 | 25 |
| Worker of Medical Area | 3 | 15 |
| Housewife | 2 | 10 |
| Other | 2 | 10 |
| Merchant | 1 | 5 |

Concomitant diseases: Thyroid; Graves' disease 2 subjects (10%) and 18 subjects (90%) none. Dermatological; 2 subjects (10%) with atopic dermatitis, 1 subject (5%) with psoriasis and 17 subjects (85%) none. (See Table 6).

TABLE 6

| Concomitant Diseases | n | % |
|---|---|---|
| Thyroid | | |
| Grave's Disease | 2 | 10 |
| None | 18 | 90 |
| Dermatological | | |
| Atopic Dermatitis | 2 | 10 |
| Psoriasis | 1 | 5 |
| None | 17 | 85 |

Region of onset of vitiligo: 11 subjects (55%) presented in the upper extremities, 3 subjects (15%) in the lower extremities, 2 subjects (10%) in the head and neck, 1 subject (5%) in the thorax, 1 subject (5%) abdomen, 1 subject (5%) in the genital region and 1. subject (5%) in the buttocks, this information coincides with what the literature reports.

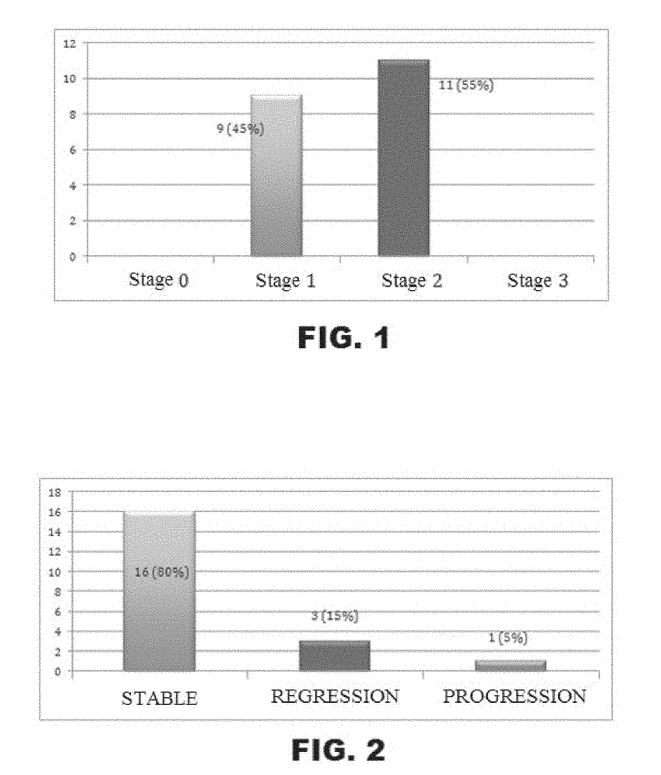

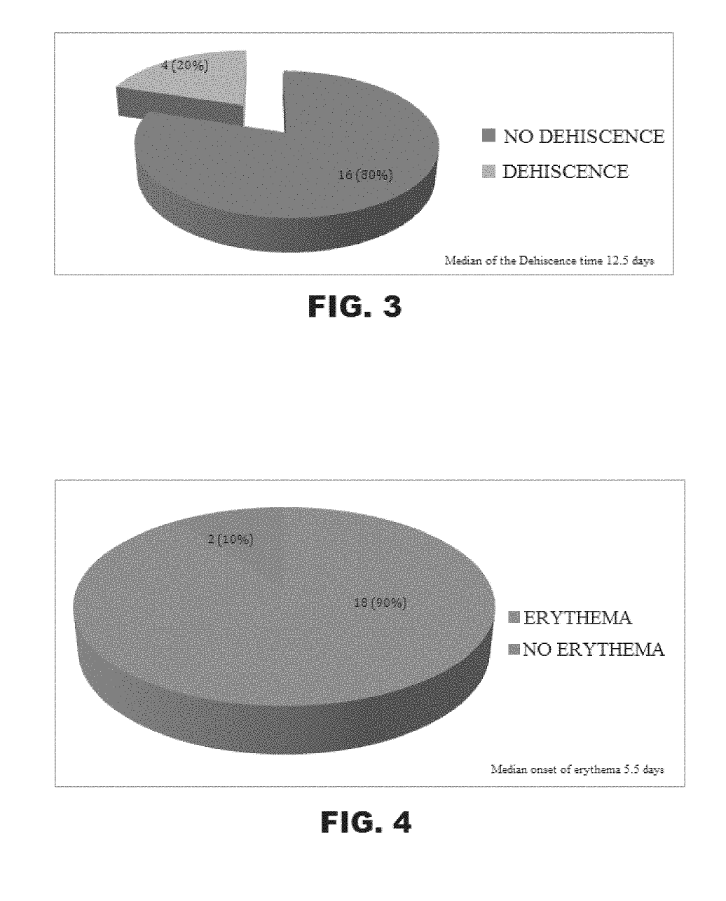

From the 20 subjects randomized, 9 (45%) were in stage I, 11 (55%) in stage II, according to the classification of the Vitiligo European Task Force (VETF): Stage 0 without depigmentation; Stage II, acromic stain with less than 30% of colorless annexes; Stage III, acromic spot with more than 30% of colorless annexes (FIG. 1). The initial status of the vitiligo according to the clinical evaluation and that referred by the subjects in the selection visit, 16 (80%) had no changes in the last year considering them as stable status; 3 (15%) reported having noticed improvement in the last year, which was considered a regression status; 1 (5%) reported that his illness had worsened in the last year, classifying it as a Progression status; (FIG. 2). A biopsy was taken in the selection visit to the 20 subjects using a spindle technique, including the acromic, transition area and normal skin area; 60% were in upper extremities, 25% in thorax, the remaining 15% in other regions. 4 patients (20%) presented wound dehiscence without infection data associated with a median of the dehiscence time of 12.5 days. (FIG. 3)

During the treatment, 18 subjects (90%) presented erythema of acromic spots, which translates as a clinical response to the treatment with a median onset of the erythema of 5.5 days. From these subjects, 3 (15%) had islands of pigment in the acromic spots, which translates as disease regression (improvement) with a median of appearance of 30 days. (FIG. 4).

In relation to the adverse events observed during the treatment, flu syndrome, drowsiness and pruritus were the most frequent (60%), predominantly of slight intensity. Other events observed with relative frequency were headache, asthenia and adynamia, also of slight intensity and rarely moderate. There were events such as anxiety, polyphagia and constipation from slight to moderate intensity, prevailing the slight intensity; polydipsia and transvaginal bleeding were uncommon events (3 and 1 subject respectively) but of moderate intensity. One subject presented slight intensity diarrhea. These events are probably related to the research product; however, a study with a greater number of samples and a comparative study is required to establish a causal relationship. During treatment, the following comorbidities were observed: 2 candidosis (oral and vaginal, respectively) and a urinary tract infection not related to the investigational product. (See Tables 7 and 8).

TABLE 7
Adverse events during the treatment with T cell Modulator

| | Slight | | Moderate | | Severe | n | |
|---|---|---|---|---|---|---|---|
| | (%) | | (%) | | (%) | (%) | |
| Flu Syndrome | 11 | (55) | 1 | (5) | — | 12 | (60) |
| Drowsiness | 9 | (45) | 3 | (15) | — | 12 | (60) |
| Pruritus | 11 | (55) | 1 | (5) | — | 12 | (60) |
| Headache | 8 | (40) | 2 | (10) | — | 10 | (50) |
| Asthenia | 7 | (35) | 2 | (10) | — | 9 | (45) |
| Adynamia | 6 | (30) | 2 | (10) | — | 8 | (40) |
| Anxiety | 4 | (20) | 1 | (5) | — | 5 | (25) |
| Polyphagia | 2 | (10) | 2 | (10) | — | 4 | (20) |

| | | | | | | |
|---|---|---|---|---|---|---|
| Constipation | 3 | (15) | — | — | 3 | (15) |

| | | | | | | |
|---|---|---|---|---|---|---|
| Polydipsia | — | 3 | (15) | — | 3 | (15) |
| Transvaginal Bleeding | — | 1 | (5) | — | 1 | (5) |

| | | | | | | |
|---|---|---|---|---|---|---|
| Diarrhea | 1 | (5) | — | — | 1 | (5) |

TABLE 8
Comorbidities During Treatment

| | Slight | Moderate | Severe | n |
|---|---|---|---|---|
| | (%) | (%) | (%) | (%) |
| Candidosis * | 1 (5) | 1 (5) | — | 2 (10) |
| Urinary tract | 1 (5) | 1 (5) | — | 2 (10) |
| infection | | | | |

* Oral Infection and another with vaginal infection

From the 20 subjects admitted, 4 were withdrawn from the study due to the following causes:

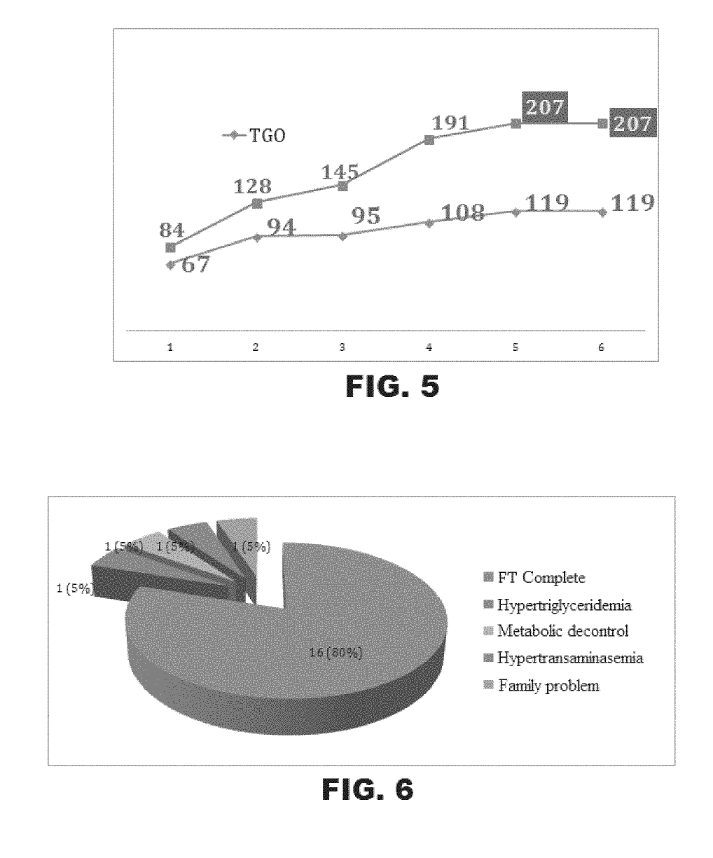

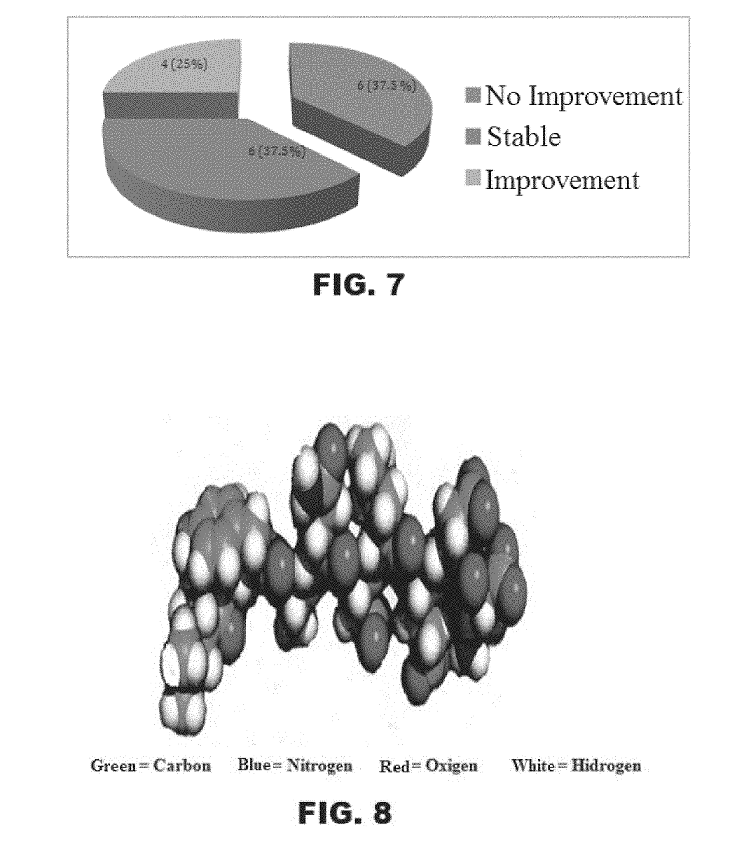

1.—Hypertransaminasemia grade H: the subject at his entrance to the study had TGO 67 and TGP 84 UI/L levels that were rising until exceeding 3.5 times the normal upper limit (TGO 119 and TGP 207 UI/L) (FIG. 5). The investigational product was discontinued in the 7th shot, hepatic ultrasound was requested, which reported slight hepatic steatosis and the monitoring of transaminases was continued, observing a decrease and normalization therefo after 50 days of suspension of the investigational product. This event is related to the investigational product. The subject did not present any clinical symptoms during his evolution and this laboratory alteration was observed in other subjects without having clinical significance and all were grade I, they did not require withdrawal of the medication.

2.—Metabolic decontrol: the subject received 5 doses of the investigational product presenting glucose in fasting altered, he was asked for HbA1C to establish a pre-existing diabetes reporting 6.7 mg/dl for which the diagnosis of DM2 was made which required the starting of the treatment. In addition, an evaluation was completed with a urine general analysis and lipid profile finding mixed dyslipidemia and proteinuria, it was excluded from the protocol as it required the initiation of management of its metabolic diseases.

3.—Hypertriglyceridemia: the investigational product was suspended after 10 doses of treatment when the subject referred triglyceride elevation in a control study carried out in its primary care center. When the results were not available, a lipid profile was requested, reporting Triglycerides of 2275 mg/dL. The subject was withdrawn since it required treatment with fibrates at high doses and hospitalization for management and surveillance, the subject refused treatment and stopped attending the center, we could only have telephone contact with the subject who reported having been hospitalized for six days without complications. Until the end of the study, the subject did not report symptoms or subsequent hospitalizations. Due to this finding and for safety, lipid profile study was indicated to all participants, and no other alarming figure was found that would indicate the removal of any subject from the study.

4.—Abandonment of Treatment: the subject received 28 doses of the investigational product, he requested his retirement from the study due to family problems. He attended the end-of-treatment visit where the corresponding laboratory studies were performed, finding TSH of 6.42 μUI/ml without a clinical picture of hypothyroidism and without presenting any change compared with the basal study. The rest of laboratories without alterations. (FIG. 6)

Other laboratory findings reported during the administration of the investigational product were: in the blood biometry, neutropenia and lymphopenia grade I without clinical repercussion; in the blood chemistry, altered fasting glucose without reaching diagnostic levels of diabetes mellitus; in the liver profile: hyperbilirrunemia grade I not clinically significant and hypertransaminasemia grade I without clinical significance except for the subject who was withdrawn from the study previously commented. Some subjects already had alterations grade I non-clinically significant in their laboratories during the selection phase. Only one subject presented constant elevation of the bilirubins from the period of selection who had no clinical significance, all the other laboratory alterations observed were transient. (See Tables 9, 10, 11),

In relation to the global response to the T-cell modulator CH11001HZ, from the 16 subjects who completed the study 6 (37.5%) presented progression of the disease interpreted as the appearance of new lesions or growth of previous lesions; 10 subjects (62.5%) had no progression of the disease and from these, 4 (40%) presented improvement of the same observed as certain degree of repigmentation. (FIG. 9)

In relation to the biopsy, incisional skin biopsies were included before and after to the administration of the investigational product in an integral manner into a capsule and labeled with consecutive numbers each. After the process of each tissue was performed, which were embedded in paraffin and blocks were made of each one, they were cut to 3 microns of thickness, after each cut they were placed on a slide and stained with Hematoxylin and Eosin; at the end the coverslips were placed. Four more cuts of each case were made at 2 microns of thickness; in each one the immunoreaction was carried out with the antibody BCL2 clone 100/D5 with dilution 1:25; the antibody CD4 clone BC/1F6, dilution 1:25; the antibody CD8 clone SP16, dilution 1:50; and the antibody HMB45 clone HMB45, 1:50 dilution. Then the microscopic examination of each one was carried out with its respective antibody with the optical microscope, using the objectives 10×, 20× and 63×. The whole biopsy was analyzed, and it was histologically divided into seven layers: stratum corneum, granular stratum, spinous layer, basal layer, papillary dermis, superficial dermis and deep dermis, wherein the quantity of lymphocytes of CD4, CD8 and their location in the Layers of skin was described. In addition, the location of the lymphocytes described in the dermis was reported, either around annexes or around vessels; The presence of melanocytes was also described with the help of HMB45 and the presence of BCL2 positive compared to the initial biopsy. It should be noted the elevation of the lymphocytic infiltrate CD8 and CD4 after the administration of the investigational product.

The dermatological questionnaire of life quality (IQLD) was applied, which assesses the impact on the quality of life associated with the disease and the treatment received. This evaluation places the subjects in four groups: no affection, little, moderate and a lot of affection. Prior to the administration of the treatment, 65% of the subjects belonged to the group without affection, at the end of the treatment 68%; 25% of the subjects prior to the administration were in the group of little affection, this percentage did not present modification. The remaining 10% of the subjects were grouped into those of moderate affection prior to the administration of the investigational product, which was reduced at the end of the administration of the treatment to 6.3%.

### CONCLUSIONS

Being a study whose primary objective was to evaluate the safety of the T-cell modulator (CH1001HZ) in subjects with vitiligo, we can conclude that the investigational product was safe for these subjects since any serious adverse event related to the investigational product was presented. Adverse events of slight and moderate intensity were presented, which did not endanger the life of the subjects, probably related to the investigational product, however, due to the fact that sample was a small and non-comparative a causal relationship cannot be determined. Within the laboratory findings, the elevation of transaminases was an event related to the administration of the investigational product, thus, surveillance of this laboratory parameter is suggested, especially in subjects with risk factors: alcoholism, use of hepatotoxic drugs and elevation previous of transaminases due to other causes.

With respect to the secondary objective that was to evaluate the response to the investigational product, a clinical response was observed in 90% of the patients, manifested as erythema of the acromic spots; 62.5% did not present progression of the disease, of which 40% presented frank repigmentation data.

From the histopathological analysis of the samples, it is worth highlighting the increase of lymphocytes CD4 and CD8 after drug administration, although the clinical translation of this finding cannot be yet established.

Finally, it is important to mention that the studies carried out up to date on the purification of the T cell modulator indicate that there are 150 molecules of polypeptides, each polypeptide has 44 amino acids on average and from the 44 amino acids, 10 are a common region.

About the common region, there are 2 large groups that differ only in one amino acid and by studying the sequence of the amino acids using the BLAST technique a similar protein containing the sequence of the common region was not found in nature, so there is a very great difficulty to get the receptor of the cell modulator, coupled with the lack of an antibody that recognizes said TCM.

Next, the amino acid sequence of a molecule of the T-cell modulator which was sequenced and the three-dimensional structure of the common region of the transfer factor is indicated.

The amino Acids Sequence (Molecula Standard 44aa)

MA [symbol] NEINDEIKDVKTTNDELDQYLLALLYAQDLEDN

The sequence conserved is indicated in italics and bold letters. Also, in FIG. 8 the three-dimensional structure of said conserved sequence is shown.

1. Potentiated T cell modulator (TCM) designed for the treatment of vitiligo, said TCM is characterized in that: it is obtained by means of a dialyzable extract of leukocytes from leukocyte cells containing polypeptides equal to or less than 10,000 daltons, whose specific source is the spleen of sharks, and in that it has the ability to reactivate the cells of the immune system of the individual, providing the body with the ability to reactivate the cells responsible for the pigmentation of the skin, that is, the melanocytes, and balance the production of melanin in the skin by said cellular activation, wherein said TCM has a potency of 10¹² leukocytes×mm³ (meaning by potency the amount of leukocytes and quality of smooth, round and innocuous cells), whose power is the amount necessary for the excitation of leukocytes and optimization of chemical signals for the production of white blood cells, which in turn produce melanocytes and the latter melanin,

2. The potentiated T cell modulator according to claim 1, wherein said TCM helps neurotransmitters to re-establish the chemicals signaling that travel from the brain to the bone marrow by coupling to the chemical signals of the neurotransmitters in order to potentiate the signal, which manages to establish the correct chemical signaling to the melanocytes and thus “order” them to start producing melanin.

3. The T cell modulator potentiated according to claim 2, wherein it order the melanocytes to restore the correct levels of melanin production to achieve pigmentation of the areas depigmented by the low amount of melanin and therefore cause the regression of the vitiligo disease.

4. The potentiated T cell modulator according to claim 1, wherein said TCM has as main function to help the production and correct restoration of white cells and the excitation thereof, in order to achieve the optimal functioning of the immune system.

5. The potentiated T cell modulator according to claim 1, wherein it is specifically designed for the treatment of the disease known as vitiligo by administering the cytosine (s) suitable for the activation of melanocytes that will produce melanin and thus repigment the areas depigmented by the lack of melanin.

6. The potentiated T cell modulator according to claim 1, characterized in that it promotes the immediate appearance of erythema in the depigmented area, which indicates the action of proinflammatory cytokines (IL-2, IFNγ and TNF α), initiating a chain reaction of the chemical signals that lead to the regulation of the immune system, especially the cytotoxic T lymphocytes that act on the melanocytes.

7. The potentiated T cell modulator according to claim 1, wherein said TCM is in a powder presentation, to be easily transported and stored, without requiring refrigeration.

8. The potentiated T cell modulator according to claim 7, wherein said TCM powder by its immunomodulatory characteristics, does not interfere with traditional treatments to certain diseases.

9. Use of a potentiated T cell modulator (TCM) with a potency of 10¹² leukocytes×mm³ whose specific source is the spleen of selacimorphs or sharks, and is obtained by a dialyzable extract of leukocytes from leukocyte cells containing polypeptides equal to or less than 10,000 daltons, or its pharmaceutically acceptable salt in the manufacture of a medicament for use in the treatment of the disease known as vitiligo.

10. The use according to claim 9, wherein patients with vitiligo, treated with said TCM or its salt, first stop the advancement of skin depigmentation in the first months, to subsequently begin to produce islands of pigment in its acromic spots, which translates into a regeneration of the depigmented parts around the sixth month.

11. The use according to claim 9, further characterized in that patients with vitiligo treated with said TCM or its salt did not present any serious adverse event related thereto.
